(19) United States
(12) Patent Application Publication
(10) Pub. No.: US 2007/0154556 A1
(43) Pub. Date: Jul. 5, 2007

(54) Injectable gel-type bone-repairing material and preparing method thereof

(76) Inventors: Fang Xu, Hangzhou City (CN); Mianli Pan, Hangzhou City (CN)

Correspondence Address: JACOBSON HOLMAN PLLC, 400 SEVENTH STREET N.W., SUITE 600, WASHINGTON, DC, 20004, US

(21) Appl. No.: 11/649,849

(22) Filed: Jan. 5, 2007

Related U.S. Application Data
(63) Continuation of application No. PCT/CN05/00977, filed on Jul. 4, 2005.

(30) Foreign Application Priority Data
Jul. 22, 2004 (CN) 200410053245.7

(51) Int. Cl. A61K 38/18; A61K 9/14

(52) U.S. Cl. 424/486; 514/12; 514/54

(57) ABSTRACT

The present invention provides an injectable gel-type bone-repairing bioactive material and its preparation method. Each unit dose of said material is composed of 1 ml of component A and 45 to 55 mg of component B, wherein each milliliter sterile saline of component A contains: 10 to 40 mg alginate, 0.1 to 1 mg bone morphogenetic protein, and 10 to 20 mg stabilizer. In the component B, each milligram (mg) of component B contains: 0.0498 to 0.1476 mg aqueous-indissolvable calcium compound, 0.0498 to 0.2953 gluconolactone, 0.0040 to 0.0159 mg polyvinylpyrrolidone and some bulking agents. Said material has good biocompatibility, which can be applied simply and safely. It can be implanted into a specific treatment site of orthopedic patients without operation. Animal experiments show that the osteogenesis activity of said material is comparable to those solid bone-repairing materials containing identical doses of bone morphogenetic protein, which need to be implanted by surgery. It is applicable for reparation of bone fracture, bone nonunion, and bone defect, as well as for the treatment of diseases in orthopedic surgery and dental surgery.

This application is a continuation application of the PCT application PCT/CN2005/000977, filed on Jul. 4, 2005 and published in Chinese.

### FIELD OF THE INVENTION

The present invention relates to medical biomaterial technology. More specifically, the present invention provides an injectable bone-repairing bioactive material capable of forming a gel and its preparation method.

### BACKGROUND OF THE INVENTION

(1) Injectable Gel-type Drug Delivery System(DDS)

According to an effective drug delivery system, a drug and a biodegradation material are combined, and injected into a specific treatment site inside a patient's body, wherein the compound solidifies to form a gel and releases the drug to achieve the therapeutic effect. This DDS is convenient to use and can prolong the effective time of the drug in the patient's body. In addition, the DDS reduces the drug dosage, as well as avoids or-reduces its side effect. Implanting the drug into a patient's body by injection route also reduces the suffering of the patients.

(2) Bone repairing bioactive material

A bone morphogenetic protein (BMP) is a group of cell growth factors with potent osteoinductive activity, which can induce undifferentiated mesenhymal stem cells to differentiate into osteoblast and proliferate to form cartilage and new bone. In general, BMP is combined with various carriers to prepare different types of bone-repairing materials. These bone-repairing materials can be used for the reparation of bone fracture, bone nonunion, bone defect, as well as for the treatment of diseases in orthopedic surgery and dental surgery.

The existing bone repairing materials should be implanted into the site of an injury through a surgical method, which requires complicated operation that is expensive and causes more painful suffering to the patients. Moreover, operation implantation is not fit for the patients with clinically most frequently occurred closed fracture or for the orthopedics patients who do not need surgery.

### SUMMARY OF THE INVENTION

An object of the present invention is to provide an injectable gel-type bone-repairing bioactive material.

Another object of the present invention is to provide a method for the preparation of said bone repairing bioactive material.

The injectable gel-type bone-repairing bioactive material of this invention is characterized in that each utility dosage comprises 1 ml of component A and 45˜55 of mg component B, wherein

the ingredients and amounts of component A are that, for each milliliter distilled water, there is provided:

| | | |
|---|---|---|
| Alginate | 10˜40 | mg |
| Bone morphogenetic protein | 0.1˜1 | mg |
| Stabilizer | 10˜20 | mg. |

The ingredients and amounts of component B are that each milligram of component B contains:

| | |
|---|---|
| Aqueous-indissolvable calcium compound | 0.0498-0.1476 mg |
| Gluconolactone | 0.0498-0.2953 mg |
| Polyvinylpyrrolidone | 0.0040-0.0159 mg |
| Bulking agent | remainder. |

The above-mentioned component A can be a lyophilized product. The component B is granules that pass through a 60-mesh sieve.

The preparation method of the injectable gel-type bone repairing bioactive material comprises the following steps:

1) 1˜4 g of alginate is dissolved in 100 ml water, then 0.1˜1 mg of BMP and 10-20 mg of stabilizer are added to each milliliter of the alginate solution. The solution is divided into 1 ml units. The units of the solution are lyophilized to produce the component A.

2) 500˜1500 mg of aqueous-indissolvable calcium compound and 500˜3000 mg of gluconolactone are mixed, then the mixture is diluted up to 10 g with the stabilizer, and thoroughly blended.

3) 1˜2 ml of 4˜8% adhesive, Polyvinylpyrrolidone, is added to the mixture from the step (2). The mixture is moistened, thoroughly mixed and concocted to form an ointment. The ointment is extruded through a 20-mesh sieve to form granulates, which are dried at 80° C. The dried particles pass through a 60-mesh sieve to produce the component B.

4) The components A and B are sterilized with Cobalt-60 (⁶⁰Co), respectively. The exposure dose is 6 Kgy;

5) The components A and B are packaged separately.

From foregoing description of the present invention, the amounts of each component are consistent with the requirement of the composition.

In the present invention, said alginate is sodium alginate or potassium alginate.

In the present invention, said stabilizer is mannitol, sorbitol, polyethylene glycol, recombination or extraction of human albumin.

In the present invention, said aqueous-indissolvable calcium compound is CaCO₃, CaSO₄, or hydroxyapatite ceramic.

In the present invention, said bulking agent is mannitol or sorbitol.

In the present invention, said sodium alginate (alginate) was purchased from Dalian Yaweite Biological Co., Ltd. (Dalian, China); BMPs were either the natural bone morphogenetic protein extracted from animal bones or recombination bone morphogenetic protein of eukaryotic expression or prokaryotic expression produced by a genetic engineering method. For instance, lyophilized powder of recombination BMP was produced by Hangzhou East-China Pharmaceutical Group Gene-tech Institute (Hangzhou, China). Gluconolactone was purchased from Sigma. Polyvinylpyrrolidone was purchased from Shanghai Boao Bio-tech Co., Ltd (Shanghai, China). Hydroxyapatite ceramic was purchased from Merck. Human albumin was purchased from Sichuan Yuan-da-shu-yang Pharmaceutical Co., Ltd; PEG, CaCO₃, CaSO₄, sorbitol and mannitol are all analytical reagents.

In the present invention, said bone-repairing bioactive material is applicable for the reparation of bone fracture, bone nonunion, bone defect, as well as for the treatment of diseases in orthopedic surgery and dental surgery.

Before use, lyophilized component A is dissolved with 1 ml of sterile saline, and inhaled in a syringe. According to treatment needs, the component B is used in proportion of 1 mg moistened with 1 ul of sterile saline, blend with component A in the above syringe to produce a uniformly mixed suspension, and injected in the treatment site where the repair is needed. After a while, the suspension would form a gel at the treatment site. In the patient's body, the BMP in the gel is slowly released to produce the osteoinductive effect. The working mechanism of this composition is that: the sodium alginate in component A is a Ca²⁺-mediated gelling agent. The gluconic acid slowly released by hydrolyzing of gluconolactone in component B can regulate the release of Ca²⁺ from aqueous-indissolvable calcium compound. The released Ca²⁺ reacts with sodium alginate to form a gel and immobilize the BMP in the specific site.

This invention relates to an injectable gel-type bone repairing bioactive material and its preparative method, the positive effect is that, once the BMP and the carriers are injected into the treatment site in a liquid form, a gel-type DDS would spontaneously develops after a while, which immobilizes the BMP within the treatment site, and induces the osteogenesis. This bone-repairing material has excellent compatibility. The carriers used in the present invention show no hazardous effect and when injected into the body, no adverse effect is observed. The simple injection administration avoids the surgical trauma and relieves the pain of the patients. Moreover, according to the therapeutic demand, the dosage can be adjusted and the administration can be repeated. Animal experiments show that the osteogenesis activity of said material in the present invention is comparable to those solid bone-repairing materials, and the clinical effect is positive and definite.

### DETAILED EMBODIMENT

Further description of the present invention is provided in combination with the following examples.

### Example 1

1) 1.5 g of sodium alginate was weighted, and dissolved in 100 ml of water to obtain a 1.5% solution. 10 mg of BMP was added to such solution, and then 2 g of mannitol was added. The solution was thoroughly mixed, divided and lyophilized to produce the component A;

2) 367 mg of CaCO₃ and 436 mg of gluconolactone were blended with 4197 mg of mannitol, and thoroughly mixed;

3) 800 ul of 8% PVP was added to the mixture from step 2). Then the mixture was moistened, blended, and concocted to form an ointment. The ointment was extruded through a 20-mesh sieve to form granulates, which were dried at 80° C. The dried particles were passed through a 60-mesh sieve to produce the component B;

4) The components A and B were sterilized with Cobalt-60 (⁶⁰Co), respectively. The exposure dose was 6 Kgy;

5) The components A and B were packaged separately.

### Example 2

1) 1.5 g of sodium alginate was weighted, and dissolved in 100 ml of water to obtain a 1.5% solution. 50 mg of BMP was added to the solution, and then 1.5 g of mannitol was added. The solution was thoroughly mixed, divided and lyophilized to produce the component A;

2) 440 mg of CaSO₄ and 436 mg of gluconolactone are blended with 4124 mg of mannitol, and thoroughly mixed;

3) 800 ul of 8% PVP was added to the mixture from step 2). The mixture was moistened, blended, and concocted into an ointment. The ointment was extruded through a 20-mesh sieve to form granulates, which were dried at 80° C. The dried particles were passed through a 60-mesh sieve to produce the component B;

4) The components A and B were sterilized with Cobalt-60 (⁶⁰Co), respectively. The exposure dose was 6 Kgy;

5) The components A and B were packaged separately.

### Example 3

1) 1.5 g of sodium alginate was weighted, and dissolved in 100 ml of water to obtain a 1.5% solution. 100 mg of BMP was added to such solution, and then 1.5 g of mannitol was added. The solution was thoroughly mixed, divided and lyophilized to produce the component A;

2) 220.2 mg of CaCO₃ and 261.6 mg of gluconolactone were blended with 2518.2 mg of mannitol, and thoroughly mixed;

3) 480 ul of 8% PVP was added to the mixture from step 2). Then the mixture was moistened, blended, and concocted to form an ointment. The ointment was extruded through a 20-mesh sieve to form granulates, dried at 80° C. The dried particles are passed through a 60-mesh sieve to produce the component B;

4) The components A and B were sterilized with Cobalt-60 (⁶⁰Co), respectively. The exposure dose was 6 Kgy;

5) The components A and B were packaged separately.

### Example 4

1) 4 g of sodium alginate was weighted, and dissolved in 100 ml water to obtain 4% a solution. 100 mg BMP was added to 100 ml of such solution, and then 2 g of mannitol was added. The solution was thoroughly mixed, divided and lyophilized to produce the component A;

2) 368.4 mg of hydroxyapatite ceramic and 1307.3 mg of gluconolactone were blended with 3324.3 mg of mannitol, and thoroughly mixed;

3) 800 ul of 8% PVP was added to the mixture from step 2). Then the mixture was moistened, blended, and concocted to form an ointment. The ointment was extruded through a 20-mesh sieve to form granulates, which were dried at 80° C. The dried particles were passed through a 60-mesh sieve to produce the component B;

4) The components A and B were sterilized with Cobalt-60 (⁶⁰Co), respectively. The exposure dose was 6 Kgy;

5) The components A and B were packaged separately.

### Example 5˜9

According to the method of Example 1, but select a formula with different ingredient and proportion, listed as following:

| Example | | | | |
|---|---|---|---|---|
| 5 | 6 | 7 | 8 | 9 |

Component A/ml:

| | | | | | |
|---|---|---|---|---|---|
| Alginate | Potassium | Sodium | potassium | Sodium | potassium |
| | alginate | alginate | alginate | alginate | alginate |
| | 10 mg | 20 mg | 30 mg | 40 mg | 20 mg |
| Bone | 0.1 mg | 0.5 mg | 1.0 mg | 1.0 mg | 0.5 mg |
| morphogenetic | | | | | |
| protein | | | | | |
| Stabilizer | sorbitol | Polyethylene | human | human | mannitol |
| | 10 mg | glycol | albumin | albumin 15 mg | 20 mg |
| | | 20 mg | 20 mg | | |

Component B/mg:

| | | | | | |
|---|---|---|---|---|---|
| Aqueous-indissol | calcium | calcium | Hydroxyapatite | Hydroxyapatite | Hydroxyapatite |
| vable calcium | carbonate | sulfate | ceramic | ceramic | ceramic |
| compound | 0.05 mg | 0.1 mg | 0.15 mg | 0.15 mg | 0.15 mg |
| Gluconolactone | 0.05 mg | 0.15 mg | 0.15 mg | 0.1 mg | 0.05 mg |
| Polyvinylpyrroli- | 0.016 mg | 0.08 mg | 0.016 mg | 0.004 mg | 0.004 mg |
| done | | | | | |
| Bulking agent | Sorbitol | Mannitol | Mannitol | Sorbitol | Mannitol |
| | Remains | Remains | Remains | Remains | Remains |

### Example 10

### Cytotoxicity Experiment

The components A and B from Examples 1 and 4, in ratio of 1 ml of component A: 50 mg of component B, were blended to obtain a suspension of 3 ml, and spread in a T-25 cell culture flask. The culture was placed in an incubation with 5% CO₂ at 37° C. for 30 minutes. After solidification, 10 ml of 1640 culture solution was carefully added to the flask. The culture was placed in the incubator to lixiviate for 24 hours. Then, the culture solution was taken out, centrifuged at 2000 g, and filtered with 0.22 um of millex. The obtained filtrate was a leaching liquor. The leaching liquor is diluted with an equal volume of 1640 culture solution to be used for the in vitro cytotoxicity assay according to the relevant regulation of GB/T16886.5.2003. The evaluation results are shown as follows:

Evaluation criterion (L929 cell is used for assay):

| | | Relative | | |
|---|---|---|---|---|
| | | growth rate of | | |
| | | cell (RGR) | | |
| Cell intoxication level | Cell morphous | range | grade | Evaluation result |
| Innocuity (−) | Eumorphism | ≧100 | 0 | Pass |
| | Shuttle or irregular | | | |
| | triangle shape cell, | | | |
| | adherence growth well, | | | |
| | cell edge regularity. | | | |
| | (excellent) | | | |
| minor (±) | Cell adherence growth | 75-99 | 1 | Pass |
| | well, minority cell turn | | | |
| | round, float dead cell | | | |
| | sparsely visible, | | | |
| | (good) | | | |
| modest (+) | Cell adherence | 50-74 | 2 | In combination |
| | undergrowth, more | | | with morphous |
| | than ⅓ cell turn | | | analysis to get a |
| | round, float dead cell | | | comprehensive |
| | visible (bad) | | | evaluation |
| severe (++) | Cell generally | 25-49 | 3 | Fail |
| | inadherent, float dead | 1-24 | 4 | |
| | cell more than 90 | 0 | 5 | |
| | percent of total cell | | | |
| | (worse) | | | |

Results:

| | | | | | | Cell | |
|---|---|---|---|---|---|---|---|
| Observation | Batch of | Cell | Mean of cell | RGR | RGR | intoxication | |
| period (day) | group | morphous | growth (%) | (%) | grade | level | Evaluation |
| 7 | Normal cell | Excellent | 37.6 | | | | |
| | control | | | | | | |
| | Gel injection | Good | 28 | 75% | 1 | ± | Pass |
| | (Example 1) | | | | | | |
| 7 | Normal cell | Excellent | 25.6 | | | | |
| | control | | | | | | |
| | Gel injection | Good | 20.4 | 80% | 1 | ± | Pass |
| | (Example 4) | | | | | | |

The results of cytotoxicity experiment were complete satisfactory, which support that the bone-repairing material has excellent biocompatibility, and is innocuous and safe.

### Example 11

### Ectopic Osteogenetic Activity Experiment

Reagents and materials: 1.5% sodium pentobarbital, 75% alcohol, 0/5# sutural line, 15# operating knife blade, hemostatic forceps, a suture needle, 1 ml syringe; 18-22 g ICR mice with same sex.

Operation Procedures:

1. Control Group (The Composite of BMP, gelatin and lecithin): The mice were anesthetized by 1.5% sodium pentobarbital. The left hind limbs were shaved and disinfected with alcohol. A 0.5-cm incision was cut on the epiderm of the muscle lacune. The skin was separated with hemostatic forceps. The muscles were blunt dissected to exposure muscle lacune. The composite containing 0.1 mg of recombination human BMP (rhBMP-2) was implanted, and the incision was sutured.

2. The Treatment group (the material of this invention): The mice were anesthetized with 1.5% sodium pentobarbital, injected with a mixed suspension of 1 ml component A and 50 mg component B into the muscle lacune of the hind limbs. Each of the mice was injected with a 0.1 ml of gel-type bone-repairing material containing 1 mg/ml rhBMP-2.

3. After 21 days, mice were anatomized, the fresh bone was taken out and weighed.

The osteogenesis activity is defined as the weight of new bone produced due to inducement of each milligram of rhBMP-2. For instance, when 1 mg rhBMP-2 induces the formation of 1000 mg of new bone, the osteogenesis activity is 1000U.

Experimental results listed below: (unit of new bone weight is mg)

| | | | | | | | | | Mean of | |
|---|---|---|---|---|---|---|---|---|---|---|
| | | | | | | | | | new | |
| | Weight of new bone | | | | | | | | bone | Osteogenesis |
| Group | 1 | 2 | 3 | 4 | 5 | 6 | 7 | 8 | weight | activity |
| control | 407 | 184 | 192 | 93 | 103 | 234 | 370 | 364 | 243 ± 123 | 2430 ± 1230 |
| treatment | 258 | 421 | 260 | 515 | 347 | 583 | 446 | 391 | 394 ± 118 | 3940 ± 1180 |

The results show that the gel-type bone-repairing material of this invention has excellent osteogenesis activity.

We claim:

1. An injectable gel-type bone-repairing bioactive material, characterized in that, each dose of the material comprises 1 ml of component A and 45˜55 mg of component B, wherein
the component A comprises: 1 ml of distilled water, 10-40 mg of alginate, 0.1-1 mg of bone morphogenetic protein, 10-20 mg of stabilizer; and
each 1 ml of the component B comprises: 0.0498-0.1476 mg of aqueous-indissolvable calcium compound, 0.0498-0.2953 mg of gluconolactone, 0.0040-0.0157 mg of polyvinylpyrrolidone, and bulking agent.

2. The material of claim 1, wherein the alginate is sodium alginate or potassium alginate.

3. The material of claim 1, wherein the stabilizer is mannitol, sorbitol, polyethylene glycol, recombination or extraction human albumin.

4. The material of claim 1, wherein the bone morphogenetic protein is the natural bone morphogenetic protein extracted from animal bone or recombination bone morphogenetic protein of eukaryotic expression or prokaryotic expression produced by gene engineering method.

5. The material of claim 1, wherein the aqueous-indissolvable calcium compound is CaCO₃, CaSO₄, or hydroxyapatite ceramic.

6. The material of claim 1, wherein the bulking agent is mannitol or sorbitol.

7. The material of claim 1, wherein the component A is a lyophilized product.

8. The material of claim 1, wherein the component B is granules passed through a 60-mesh sieve.

9. A method for preparing an injectable gel-type bone-reparing bioactive material, comprising:
a) dissolving 1˜4 g of alginate in 100 ml of water to form an alginate solution, then adding 0.1˜1 mg of BMP and 10-20 mg of a stabilizer in each milliliter of the alginate solution, dividing the solution into 1 ml units and lyophilizing the units of the solution to provide a component A;
b) mixing 500˜1500 mg of an aqueous-indissolvable calcium compound and 500˜3000 mg of gluconolactone, then diluting the mixture up to 10 g with a bulking agent, and blending the mixture until it becomes homogeneous;
c) adding 1˜2 ml of 4˜8% polyvinylpyrrolidone to the mixture from step (b), moistening the mixture, thoroughly mixing and concocting the mixture to form an ointment, extruding the ointment through a 20-mesh sieve to form granulates, drying the granulates at 80° C. to provide dried particles, and passing the dried particles through a 60-mesh sieve to provide a component B; and
d) sterilizing the components A and B with Cobalt-60 (⁶⁰Co), respectively with an exposure dose of 6 Kgy.

10. The method of claim 9 further comprising the step of separately packaging the components A and B.

11. The method of claim 9, wherein the aqueous-indissolvable calcium compound is CaCO₃, CaSO₄, or hydroxyapatite ceramic.

12. An injectable gel-type bone-repairing bioactive material, characterized in that, each dose of the material comprises component A and component B, wherein
the component A comprises: distilled water, alginate, bone morphogenetic protein, stabilizer; and
the component B comprises: aqueous-indissolvable calcium compound, gluconolactone, polyvinylpyrrolidone, and bulking agent.

13. The material according to claim 12 comprising 1 ml of component A and 45-55 mg of component B.
